(19) United States
(12) Patent Application Publication
(10) Pub. No.: US 2024/0325108 A1
(43) Pub. Date: Oct. 3, 2024

(54) DRUG-REFILLABLE, BIOCOMPATIBLE, AND BIODEGRADABLE TISSUE MARKERS AND METHODS OF MAKING AND USING THE SAME

(71) Applicant: C.R. Bard, Inc., Franklin Lakes, NJ (US)

(72) Inventors: Aaron Blank, Chandler, AZ (US); Hiep Q. Do, Chandler, AZ (US)

(21) Appl. No.: 18/194,007

(22) Filed: Mar. 31, 2023

(51) Int. Cl. A61B 90/00; A61L 31/04; A61L 31/14

(52) U.S. Cl. CPC A61B 90/39; A61L 31/041; A61L 31/145; A61L 31/146; A61B 2090/3908; A61B 2090/3925; A61B 2090/3995; A61L 2300/802; A61L 2400/04; A61L 2420/02

(57) ABSTRACT

A tissue marker is provided having a permanent portion and a biodegradable portion. The permanent portion includes a material detectable by an imaging modality. The biodegradable portion surrounds the permanent portion. The biodegradable portion includes a polymer body, a coating, and a plurality of distinct structures dispersed in the polymer body. The coating covers an outer surface of the polymer body. Each of the distinct structures include an outer shell designed to open upon the application of focused ultrasound to release a therapeutic agent within the outer shell into the polymer body. Therapeutic agents may be introduced into the biodegradable portion of the tissue marker after a period of time after introduction of the tissue marker into the tissue. In addition, methods of fabricating a three-layer tissue marker are provided.

### TECHNICAL FIELD

The present disclosure relates to biocompatible and/or biodegradable tissue markers and methods of preparing and using the same.

### BACKGROUND

Numerous medical conditions require a biopsy for both diagnosis and as a form of treatment, in which a specimen or sample of tissue is resected or removed for pathological examination, tests, and/or analysis.

As tissue heals following these procedures, identifying the location of tissue resection can become increasingly challenging, yet health professionals frequently need to monitor the exact site of resection. One particularly useful approach that allows not only surface identification, but depth of tissue identification is implantation of a tissue marker. During the biopsy and/or lumpectomy procedure(s), a tissue marker may be implanted in the space created by the tissue resection to precisely mark the location. The tissue marker may be later detected during follow-up procedures or appointments to identify the original biopsy location.

Conventional tissue markers suffer from several limitations, including migration and case of visualization. Similarly, delivery of the tissue marker may be frustrated by the size and shape of the tissue marker, the space created by the resection, and by surface interactions between the marker and the delivery device. The tissue marker may irritate or cause discomfort to the implantee. Further, some tissue markers degrade over time, making localization difficult. Accordingly, there is a need to improve the physical properties of tissue markers.

### SUMMARY

In some aspects, the present disclosure provides an improved tissue marker that overcomes, at least in part, the above-noted limitations seen in conventional markers. In some aspects, the present disclosure provides a tissue marker that includes a detectable marker, such as a metal, encapsulated within an encapsulating layer. The encapsulating layer may completely or partially encapsulate the detectable marker. In some aspects, a further coating layer may overlay the encapsulation layer and/or the detectable marker. In some aspects, the tissue markers of the present disclosure include one or more therapeutic agents. In some aspects, the therapeutic agent(s) may be embedded within micro- and/or nano-particles such that release of the therapeutic agents may be controlled by the degradation of the particle material when in situ within a subject. In some aspects, the tissue markers may be filled or refilled with a therapeutic agent when in situ within a subject. Accordingly, the tissue markers of the present disclosure provide biocompatible, and biodegradable tissue markers that can further provide localized release of one or more therapeutics with the option to adjust by providing additional therapeutic to the tissue marker in situ. In some aspects, the one or more therapeutics are contained and/or added to the encapsulation layer. In some aspects, the coating layer of the tissue markers of the present disclosure may further include at least one polymer and/or at least one protein to provide a low friction surface that will not stick in a delivery device used to place the tissue marker within a subject, that decreases incidence of extrusion from the delivery device, and that prevents migration from the delivery or target site within a subject. In some aspects, the coating layer may provide soluble materials therein to inhibit and/or slow coagulation from the subject's circulatory system. In some aspects, tissue markers of the present disclosure may be formulated for a controlled degradation time with corresponding drug delivery capabilities and/or blood anti-coagulation, which collectively assist in sealing the incision and/or cavity within which the marker is placed, such as with a biopsy or lumpectomy procedure.

In some aspects, a tissue marker includes a permanent detectable marker. In further aspects, the coating layer and/or the encapsulation layer are biodegradable, such that when the tissue marker is placed in situ within a subject, the coating layer and/or the encapsulation layer degrade over time within the subject as the site of placement within the subject heals and the subject's tissue regenerates and refills the space. The biodegradable portion surrounds the permanent portion. The biodegradable portion includes a polymer body, a coating covering an outer surface of the polymer body, and a plurality of distinct structures dispersed in the polymer body. Each of the distinct structures includes an outer shell designed to open upon the application of focused ultrasound and a therapeutic agent within the outer shell.

A 1^{st} aspect, either alone or in combination with any other aspect herein, concerns a tissue marker, comprising: a permanent or semi-permanent material detectable by an imaging modality; an encapsulation layer at least partially covering the permanent or semi-permanent material comprising a cross-linked biodegradable polymer; and a coating layer at least partially covering the encapsulation layer comprising a hydrophobic polymer, a hydrophobic small molecule, a protein, a peptide, or a combination thereof.

A 2^{nd} aspect, either alone or in combination with any other aspect herein, concerns the tissue marker of the 1^{st} aspect, wherein the permanent or semi-permanent material comprises a metal or metallic compound.

A 3^{rd} aspect, either alone or in combination with any other aspect herein, concerns the tissue marker of the 1^{st} aspect or 2^{nd} aspect, wherein the permanent or semi-permanent material is selected from titanium, nitinol, stainless steel, a cobalt-chromium alloy, a titanium alloy, a magnesium alloy, or a combination thereof.

A 4^{th} aspect, either alone or in combination with any other aspect herein, concerns the tissue marker of the 1^{st} aspect, wherein the cross-linked biodegradable polymer is a swellable hydrogel or swellable non-hydrogel.

A 5^{th} aspect, either alone or in combination with any other aspect herein, concerns the tissue marker of the 1^{st} aspect or 4^{th} aspect, wherein the cross-linked biodegradable polymer is selected from silk fibrin, chitosan, carboxymethyl cellulose (CMC), polyglycerol subacetate, and/or hyaluronic acid (HA).

A 6^{th} aspect, either alone or in combination with any other aspect herein, concerns the tissue marker of the 1^{st} aspect, wherein the coating layer is selected from a plant starch, chitosan, hyaluronic acid (HA), carboxymethyl cellulose (CMC), an HA-CMC co-polymer, beeswax, paraffin wax, glycerol, polyethylene glycol (PEG), sorbitol, a polysorbate, dextran sulfate, pectin, pullulan, hydroxypropyl methyl cellulose (HPMC), sodium alginate, polyvinyl alcohol (PVA), a glyceride, lecithin, hydrogenated coconut oil, coconut oil, cocoa butter, glycerinated gelatin, hydrogenated vegetable oils, hard fats, mineral oil, cetyl alcohol, petrolatum, petroleum jelly, decanol, soft paraffin, tridecanol, dodecanol, long chain saturated fatty acids, long chain unsaturated fatty acids, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, carnauba wax, fatty acid triglycerides, fatty acid alcohols, polyethylene glycol (PEG)-fatty acid esters, PEG-surfactants with an HLB below 13, or combinations thereof.

A 7^{th} aspect, either alone or in combination with any other aspect herein, concerns the tissue marker of the 6^{th} aspect, wherein the coating layer comprises at least one of as a plant starch, chitosan, hyaluronic acid (HA), carboxymethyl cellulose (CMC), and an HA-CMC co-polymer or is a combination thereof.

An 8^{th} aspect, either alone or in combination with any other aspect herein, concerns the tissue marker of the 1^{st} aspect, wherein the permanent or semi-permanent material further comprises a protective polymer coating.

A 9^{th} aspect, either alone or in combination with any other aspect herein, concerns the tissue marker of the 8^{th} aspect, wherein the protective polymer coating comprises fibroin, chitosan, carboxylmethyl cellulose (CMC), polyglycerol sebacate, hyaluronic acid (HA), or a combination thereof.

A 10^{th} aspect, either alone or in combination with any other aspect herein, concerns the tissue marker of the 1^{st} aspect, wherein the encapsulation layer at least partially covers a housing compartment and wherein the permanent or semi-permanent material resides within a compartment of the housing compartment.

An 11^{th} aspect, either alone or in combination with any other aspect herein, concerns the tissue marker of the 10^{th} aspect, wherein the housing compartment is comprised of fibroin, chitosan, carboxylmethyl cellulose (CMC), polyglycerol sebacate, and/or hyaluronic acid (HA).

A 12^{th} aspect, either alone or in combination with any other aspect herein, concerns the tissue marker of the 11^{th} aspect, wherein the housing compartment comprises two or more compartments.

A 13^{th} aspect, either alone or in combination with any other aspect herein, concerns the tissue marker of the 12^{th} aspect, wherein at least one compartment of the housing compartment comprises an interior space filled with a hydrogel.

A 14^{th} aspect, either alone or in combination with any other aspect herein, concerns the tissue marker of the 13^{th} aspect, wherein the hydrogel has a therapeutic agent dispersed therein.

A 15^{th} aspect, either alone or in combination with any other aspect herein, concerns the tissue marker of the 12^{th} or 13^{th} aspect, wherein the hydrogel has an excipient dispersed therein.

A 16^{th} aspect, either alone or in combination with any other aspect herein, concerns the tissue marker of the 1^{st} aspect, wherein the coating layer comprises a hemostatic polymer.

A 17^{th} aspect, either alone or in combination with any other aspect herein, concerns the tissue marker of the 16^{th} aspect, wherein the hemostatic polymer comprises microporous polysaccharide hemispheres.

An 18^{th} aspect, either alone or in combination with any other aspect herein, concerns the tissue marker of the 1^{st} aspect, wherein the encapsulation layer further comprises a therapeutic agent.

A 19^{th} aspect, either alone or in combination with any other aspect herein, concerns the tissue marker of the 18^{th} aspect, wherein the therapeutic agent is present within microparticles dispersed within the encapsulation layer.

A 20^{th} aspect, either alone or in combination with any other aspect herein, concerns the tissue marker of the 19^{th} aspect, wherein the microspheres are comprised of poly-glycolic acid (PGA) poly-L-lactic acid (PLLA), polycaprolactone (PCL), poly-DL-lactic acid (PDLLA), poly(trimethylene carbonate) (PTMC), poly (ester amine)s (PEA), poly(para-dioxanone) (PPDO), poly-2-hydroxy butyrate (PHB), a polymer combination of lactic acid and glycolic acid, poly-lactic-co-glycolic acid (PLGA), and co-polymers thereof.

A 21^{st} aspect, either alone or in combination with any other aspect herein, concerns the tissue marker of the 20^{th} aspect, wherein the microparticles further comprise an antioxidant.

A 22^{nd} aspect, either alone or in combination with any other aspect herein, concerns the tissue marker of the 18^{th} aspect, wherein the therapeutic agent is present in micelles dispersed throughout the encapsulation layer.

A 23^{rd} aspect, either alone or in combination with any other aspect herein, concerns the tissue marker of the 1^{st} or 18^{th} aspect, wherein the encapsulation layer further comprises an excipient.

A 24^{th} aspect, either alone or in combination with any other aspect herein, concerns the tissue marker of the 23^{rd} aspect, wherein the excipient is an antioxidant, a surfactant, a vitamin, or a combination thereof.

A 25^{th} aspect, either alone or in combination with any other aspect herein, concerns the tissue marker of the 23^{rd} or 24^{th} aspect, wherein the excipient comprises a surfactant and an antioxidant.

A 26^{th} aspect, either alone or in combination with any other aspect herein, concerns the tissue marker of the 1^{st} aspect, wherein the coating layer comprises a first sub-part comprised of a microporous polysaccharide and an HA-CMC copolymer mixture and a second sub-part comprised of chitosan.

A 27^{th} aspect, either alone or in combination with any other aspect herein, concerns the tissue marker of the 26^{th} aspect, wherein the chitosan is a high molecular weight chitosan.

A 28^{th} aspect, either alone or in combination with any other aspect herein, concerns the tissue marker of the 26^{th} aspect, wherein the first sub-part comprises between about 10 to about 70 wt % of the coating layer.

A 29^{th} aspect, either alone or in combination with any other aspect herein, concerns a method of preparing the tissue marker of the 1^{st} aspect, comprising: surrounding the permanent or semi-permanent material with the encapsulating layer; cross-linking the encapsulating layer; applying the coating layer to the cross-linked encapsulating layer; and cross-linking the coating layer to form a three layer tissue marker.

A 30^{th} aspect, either alone or in combination with any other aspect herein, concerns a method of the 29^{th} aspect, further comprising freeze drying the three layer tissue marker.

A 31^{st} aspect, either alone or in combination with any other aspect herein, concerns a method of the 29^{th} aspect, comprising providing a protective polymer to the permanent or semi-permanent material prior to surrounding in the encapsulation layer.

A 32^{nd} aspect, either alone or in combination with any other aspect herein, concerns a method of the 31^{st} aspect, wherein the protective polymer surrounds the permanent or semi-permanent material in a first compartment of a housing compartment.

A 33^{rd} aspect, either alone or in combination with any other aspect herein, concerns a method of the 32^{nd} aspect, wherein the housing compartment includes a second compartment filled with a hydrogel.

A 34^{th} aspect, either alone or in combination with any other aspect herein, concerns a method of the 33^{rd} aspect, wherein the hydrogel comprises a therapeutic agent.

A 35^{th} aspect, either alone or in combination with any other aspect herein, concerns a method of the 29^{th} aspect, wherein the encapsulation layer includes a therapeutic agent.

A 36^{th} aspect, either alone or in combination with any other aspect herein, concerns a method to provide support to a recess or void in a subject, comprising providing an access point to a desired tissue space in a subject, placing the tissue marker of the 1^{st} aspect in the desired tissue space, and closing the access point.

A 38^{th} aspect, either alone or in combination with any other aspect herein, concerns a method of the 36^{th} aspect, further comprising injecting a solution comprising a therapeutic agent into the tissue marker.

These and additional features provided by the embodiments described herein will be more fully understood in view of the following detailed description, in conjunction with the drawings.

### BRIEF DESCRIPTION OF DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A schematically depicts a partial cut-away perspective view of an illustrative tissue marker according to one or more embodiments of the present disclosure;

FIG. 1B schematically depicts a transverse cross-sectional view of the tissue marker taken along line 1B-1B of FIG. 1C, according to one or more embodiments of the present disclosure;

FIG. 1C schematically depicts an exterior side view of the tissue marker taken along line 1C-1C of FIG. 1A, according to one or more embodiments of the present disclosure;

FIG. 2A schematically depicts a partial cut-away perspective view of another illustrative tissue marker, according to one or more embodiments of the present disclosure;

FIG. 2B schematically depicts a longitudinal cross-sectional side view of the tissue marker taken along line 2B-2B of FIG. 2A, according to one or more embodiments of the present disclosure;

FIG. 2C schematically depicts an enlargement of the tissue marker as indicated by the region 2C of FIG. 2B, according to one or more embodiments of the present disclosure;

FIG. 3 depicts a flowchart of an exemplary method of introducing a therapeutic agent into a tissue at a target site, according to one or more embodiments shown and described herein;

FIG. 4A schematically depicts a first layer of a three-layer tissue marker, according to one or more embodiments shown and described herein;

FIG. 4B schematically depicts a sub-assembly comprising first and second layers of a three-layer tissue marker, according to one or more embodiments shown and described herein;

FIG. 4C schematically depicts a three-layer tissue marker, according to one or more embodiments shown and described herein;

FIG. 4D depicts a flowchart of an exemplary method of fabricating the tissue marker, according to one or more embodiments shown and described herein; and

FIG. 5 depicts a graphical representation of the results of testing bovine blood clotting time of different formulations, according to one or more embodiments shown and described herein.

Corresponding reference characters indicate corresponding parts throughout the several views. The exemplifications set out herein illustrate at least one embodiment of the present disclosure, and such exemplifications are not to be construed as limiting the scope of the present disclosure in any manner.

### DESCRIPTION

Embodiments described herein are generally directed to tissue markers, methods of introducing the tissue markers of the present disclosure into a subject at a target site, and methods of fabricating the tissue markers as described herein. For example, a tissue marker according to the present disclosure generally includes a detectable marker that includes a material detectable by an imaging modality, such as, for instance a metallic material, an encapsulation layer that includes one or more biodegradable polymers, and a coating layer. The tissue markers described herein provide for compositions that permanently or semi-permanently mark a desired location on a subject, such as a site for a biopsy or lumpectomy. The tissue markers as set forth herein provide biocompatibility with the subject, such that no adverse reaction to the presence of the tissue marker can be expected. In further aspects, the encapsulation layer, in addition to providing support to site surrounding tissue in situ, provides a matrix within which one or more therapeutics may be loaded. In some aspects, the therapeutics can be load post-procedurally and/or pre-placement within the subject. In further aspects, one or more therapeutics may be contained within a microparticle and/or nanoparticle. In further aspects, the coating layer provides a dissolvable coating to protect the encapsulation layer and/or detectable marker prior to placement within the subject. The coating layer may include anti-coagulation compositions, as well as provide external features that prevent migration when the tissue marker is placed in situ. These and additional features and benefits are described in greater detail herein.

The removal of or excision of tissue from a subject leaves a void. Even in instances where sealing or suturing the incision points to access the tissue is barely visible, the removal of underlying tissue leaves a void. Surrounding tissue may be able to sense the space and eventually refill the void, but such takes time and in the interim, the surrounding tissue is unsupported and risks repercussive events such as collapse. Furthermore, tissue is often removed to assess or resect tissue of concern. In such instances, it can be better to overestimate the amount of tissue that needs removal in order to reduce of chances of only partial removal. For example, with cancerous or pre-cancerous tissue, the proliferative and/or anti-apoptotic nature of the cells can lead to a large volume of diseased tissue from only a small number of cells.

Obtaining a tissue sample, such as by biopsy or lumpectomy, and the subsequent examination are conventionally steps for varying processes such as assessment for the presence of cancerous or pre-cancerous cells. The information obtained from tissue diagnostic tests and/or examinations is frequently used to plan for the appropriate surgical procedure and/or other course(s) of treatment. For example, breast biopsies may be performed where a suspicious lump or swelling is noticed in a breast. After a tissue sample is taken, it may take several days or weeks before the results of the examination of the sample are obtained, and still longer before a treatment plan is determined. Similarly, if surgery is utilized to remove malignant or suspected malignant tissue, the surrounding tissue requires monitoring over significant amounts of time to ensure complete resection of all malignant or tumorigenic cells. With time allowing for healing of tissue, it is clear how tissue markers or an implanted marker within the tissue provide a means to rapidly and easily locate the original site of resected tissue. Thus, the present disclosure addresses the need for tissue markers, methods of introducing a therapeutic agent with the tissue marker into a tissue at a target site, and methods of fabricating an at least three-layer tissue marker.

In some aspects, the present disclosure concerns a tissue marker with a detectable marker, an encapsulation layer, and a coating layer. In some aspects, the encapsulation layer at least partially encapsulates the detectable marker, such as of about 20 to about 100 percent of the external surface area of the detectable marker, including about 25, 30, 35, 40, 45, 50, 55, 60, 65, 70, 75, 80, 85, 90, 95, 96, 97, 98, 99, and 99.9% encapsulation of the outer surface area of the detectable marker. In some aspects, the coating layer covers at least a portion of the encapsulation layer and/or the detectable marker, such as of about 25 to about 100% of the outer surface are of the encapsulation layer and/or the detectable marker, including about 30, 35, 40, 45, 50, 55, 60, 65, 70, 75, 80, 85, 90, 95, 96, 97, 98, 99, and 99.9% encapsulation of the outer surface area of the encapsulation layer and/or the detectable marker.

### Permanent/Semi-Permanent Material

In some aspects, the tissue marker may include a permanent or semi-permanent material. As descried herein, the encapsulation layer and/or the coating layer are designed to erode and/or dissolve as the tissue heals, allowing for the subject's own cells to eventually fill the majority of the void created by resection and/or excision of tissue within the subject. In some aspects, the tissue marker may be of a metal and/or metallic compound that is easily or readily detected through the subject's skin and/or muscle and/or tissue, such that detection is achieved without incision and/or insult to the subject. Such techniques can include X-ray, ultrasound, magnetic resonance imaging (MRI), computerized tomography (CT) and/or computerized axial tomography (CAT) scans, fluoroscopy, and/or positron emission tomography (PET) scans. In some aspects, the tissue marker includes a permanent material and/or semi-permanent material that allows for detection over a prolonged period of time, including at least 6 months, at least one year, at least 5 years, and at least 10 or more years. Such materials may include metals or metallic materials, metal compounds, metal amalgams, metal alloys, or the like. In some aspects, the tissue marker includes titanium, nitinol (nickel-titanium), stainless steel, cobalt-chromium alloys, titanium alloys, magnesium alloys, and/or other biocompatible metals or metal alloys that are detectable by the imaging process or machinery to be used. In some aspects, the material should be selected with respect to the imaging system to be used so as to avoid conflicts, such as between ferromagnetic materials and magnetic-based imaging.

In some aspects, the tissue marked may further include a biodegradable material, such that the tissue marker decreases in situ over time within the subject, ultimately to leave the permanent and/or semi-permanent portions embedded within the subject's healed tissue. Such may be desirable when the marker is to provide identification of a larger area within the subject without needing to deposit a larger amount of permanent and/or semi-permanent material. For example, suspending a permanent and/or semi-permanent material within a biodegradable material allows for the permanent and/or semi-permanent material to be dispersed over a wider area as the coating layer, encapsulation layer and biodegradable material erode and/or dissolve within the subject. It will be also appreciated that dispersing a permanent and/or semi-permanent material within the encapsulation layer may also achieve dispersal across the volume of the excised and/or resected tissue.

In some aspects, the permanent/semi-permanent material is a metal. In some aspects, the permanent/semi-permanent material may be covered with a polymer protective material to promote biocompatibility, such as a coating with fibroin, chitosan, carboxylmethyl cellulose (CMC), polyglycerol sebacate, and/or hyaluronic acid (HA).

In some aspects, the permanent/semi-permanent material is housed within a protective material, such as fibroin, chitosan, carboxylmethyl cellulose (CMC), polyglycerol sebacate, and/or hyaluronic acid (HA). In some aspects, the permanent/semi-permanent material is housed within a protective polymer to form a housing compartment. In some aspects, the housing compartment includes a protective polymer surrounding or encasing the permanent/semi-permanent material within a single layer. In other aspects, the housing compartment includes two or more separated spaces therein, with one space surrounding the permanent/semi-permanent material and a second space surrounding a hydrogel. In some aspects, the hydrogel within the second compartment can be loaded and/or re-loaded with solutions, such as with solutions with one or more therapeutics suspended and/or dissolved therein. It is a further aspect of the present disclosure that the hydrogel in the housing compartment(s) can be loaded and/or reloaded with therapeutics in situ. Such techniques can be achieve by injection directly into the hydrogel portion within the housing compartment.

### Encapsulation Layer

In some aspects, the present disclosure concerns an encapsulation layer that at least partially encapsulates the tissue marker. As described above, in some aspects, the permanent/semi-permanent component may be enclosed within a protective polymer or a housing compartment. It is accordingly to be understood that reference to encapsulation of permanent/semi-permanent material will also refer to encapsulation of the housing compartment and/or protective polymer. In some aspects, the permanent/semi-permanent component is entirely encapsulated by the encapsulation layer. In some aspects, the encapsulation layer encapsulates two or more distinct permanent/semi-permanent components that are either of the same material or of varying materials. In some aspects, the permanent/semi-permanent component may be of a limited size, such as due to material scarcity, material density, and the like. In such aspects, dispersing permanent/semi-permanent particles throughout the volume of the encapsulation layer allows for the tissue marker to effectively disperse throughout the tissue void when implanted without actually requiring full occupancy of the void itself. For example, it will be appreciated that the particles may overlap when imaged or provide sufficient proximity to each that the user can readily determine the detected tissue marker to be of the same site within the subject. Similarly, dispersing different materials within the encapsulation layer may allow for additional imaging modalities to be available to a user, thereby increasing convenience to the subject and user.

In some aspects, the encapsulation layer may include a cross-linked biodegradable polymer, including hydrogels and/or non-hydrogels. In some aspects, the polymer is swellable, such as a swellable hydrogel or swellable non-hydrogel. The encapsulation layer may be fabricated in any desired shape, such as a shape that resembles the void created by the excised or resected tissue. In some aspects, the encapsulation layer is of a material that is inert to the subject and/or non-immunogenic, such that the subject's immune system will not react to the material.

In some aspects, the encapsulation layer is of a bioabsorbable or biodegradable polymer, such as at least one of silk fibrin, chitosan, carboxymethyl cellulose (CMC), polyglycerol subacetate, and/or hyaluronic acid (HA). In some aspects, polymerization of the materials allows for increased structure, such that the encapsulation layer provide physical support to the tissue surrounding the void. It will be apparent that increasing the amount of polymerization or cross-links within the polymer's structure may increase the firmness or rigidity within the void, though such may also slow the rate of degradation in situ within the subject. While other polymer materials may be considered, such as poly glycolic acid (PGA), poly lactic acid (PLA), co-polymers of PLA and PGA (PLGA), and polycaprolactone (PCL), silk fibrin, chitosan, CMC, polyglycerol subacetate, and/or HA fail to trigger the same levels of immunoreactivity. Accordingly, these materials can reside in situ in a subjects tissue with minimal immune response, thereby allowing for increased healing times, as well as reduced effects such as reduced inflammation, irritation, redness and similar.

In some aspects, the encapsulation layer includes polymerized polymer material within the layer, such as polymerized silk fibrin, chitosan, carboxymethyl cellulose (CMC), polyglycerol subacetate, and/or hyaluronic acid (HA). It will be appreciated that the degree of polymerization and/or the selection of polymer material(s) and/or the concentration of the polymer material(s) can be selected to achieve a preferred level of elasticity and/or viscoelasticity with a desired level of firmness. In some aspects, the encapsulation layer may possess a Young's modulus, shear modulus, and/or storage modulus of from about 12 Pa to about 4 MPa.

It will be appreciated that the nature of the materials listed allows for the encapsulation layer to swell. Accordingly, in some aspects, at least a part of the coating layer will dissolve and/or degrade once the tissue marker is placed in situ within a subject. The exposure of the encapsulation layer to the tissue and/or extracellular matrix materials within the subject will cause aqueous media to swell the encapsulation layer. As such, the tissue marker allows for a delayed swelling of the encapsulation layer following insertion. Such allows a user sufficient time to close in tissue marker or suture the subject such that the tissue marker is embedded within the subject. As the encapsulation layer swell, a space or void within the subject where the tissue marker was placed is filled with the swell encapsulation layer. Swelling of the encapsulation layer thereby provides internal organ and/or tissue support from the now-filled cavity space, as well as prevents migration within the subject.

In some aspects, the encapsulation layer may be subdivided such that two or more compartments are formed. In some aspects, the compartments may be separated by different polymer compositions and/or different degrees of cross-linking therein, such that there is a difference in degradation time, modulus, swellability or combinations thereof between compartments. In some aspects, a physical barrier may isolate the compartments. In some aspects, the physical barrier is degradable and/or dissolvable. In some aspects, the physical barrier may include one or more facets of the coating layer as described further herein. In some aspects, the encapsulation layer may include a wall, wherein the wall is of the same material as the rest of the encapsulation layer but with a different level of cross-linking and/or concentration or density of biodegradable polymer therein.

In some aspects, the encapsulation layer may include one or more additional components therein. In some aspects, due to the polymerized nature of the encapsulation layer, one or more components may be suspended therein. In some aspects, as the polymer of the encapsulation layer degrades over time, the one or more components are released and/or exposed to allow for interaction and/or absorption by the subject. It is accordingly a further aspect of the encapsulation layer that the release of the one or more components suspended therein is controlled by the rate of degradation of the polymer of the encapsulation layer.

In some aspects, the one or more components may include a therapeutic agent, or two or more therapeutic agents. In some aspects, the therapeutics are suspended within the polymer of the encapsulation layer in an amorphous or crystalline form, such that degradation of the polymer surrounding them allows for their ultimate release, wherein the subject's tissue surrounding the tissue marker can absorb and benefit from the physiological effects caused by the therapeutic(s). As discussed herein, a therapeutic may further be encapsulated within the encapsulation layer polymer by being suspended and/or distributed throughout the body of nanoparticles. The materials and approaches to preparing such are discussed in more detail further herein.

In some aspects, the one or more therapeutics may be formulated such that a protective drug coating encapsulates the active compound(s).

In some aspects, the protective drug coating may include a micelle surrounding the one or more active compound(s). In some aspects, the protective drug coating retrains the active compound(s) of the therapeutic agent(s) until an external force disrupts the protective drug coating, such as a focused ultrasound. Micelles typically include an aggregate of amphipathic lipids that include a hydrophilic “head” and a hydrophobic “tail.” It will be appreciated that micelles may include single layer and bi-layer micelles. Methods of loading micelles are understood in the art, such as though sonication, microfluidic mixing and so forth. The average size of each of the micelles may be of 1.1 micrometers plus or minus 0.39 micrometers.

In some aspects, the therapeutic may be included within the body of a microparticle. Microparticles may be prepared the evaporation of a solvent with a bioabsorbable/biodegradable polymer and at least one therapeutic therein. In some aspects, the solvent is of dichloromethane (DCM) or ethyl acetate (EtOAc). Polymers may include a network of a poly-glycolic acid (PGA) and a poly-L-lactic acid (PLLA). Other bioabsorbable polymers that can be utilized in combination or alone for the microparticles include polycaprolactone (PCL), poly-DL-lactic acid (PDLLA), poly(trimethylene carbonate) (PTMC), poly (ester amine)s (PEA), poly(para-dioxanone) (PPDO), poly-2-hydroxy butyrate (PHB), and co-polymers with various ratios thereof. In some aspects, the bioabsorbable polymer may include, either alone or in combination with other bioabsorbable polymers, a polymer combination of lactic acid and glycolic acid, poly-lactic-co-glycolic acid (PLGA). Those skilled in the art will appreciate that PLGA can be of varying percentages of lactic acid and glycolic acid, wherein the higher the amount of lactide units, the longer the polymer can last in situ before degrading. Additional tunable properties with PLGA concern the molecular weight, with higher weights showing increased mechanical strength. In some aspects, the polymer microparticle is also loaded or embedded with an antioxidant, such as BHT.

The size range of the microparticles may be of 10 micrometers up to and including 200 micrometers. In some embodiments, the average size of each of the microparticles is from 60 nanometers to 70 nanometers. The density of the therapeutic agent or polymer microparticle within the microparticle is of from about 0.1 to 10 μg/mm². In certain aspects, the therapeutic agent is provided at a density of from about 0.5 to about 5 μg/mm². In some aspects, the dose density of the therapeutic agent(s) within each polymer microparticle can vary from about 0.1 to about 10 μg/mm², including about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, and 9.9 μg/mm². In some aspects, the drug dose density is of about 0.5 to about 5 μg/mm².

The therapeutic may include one or more of paclitaxel, sirolimus (rapamycin), daunorubicin, 5-fluorouracil, doxorubicin, sunitinib, sorafenib, irinotecan, bevacizumab, cetuximab, biolimus (biolimus A9), everolimus, zotarolimus, tacrolimus, tadalafil, sildenafil, dexamethasone, prednisolone, corticosterone, 5-fluorouracil, cisplatin, vinblastine, lidocaine, bupivacaine, and all analogs, derivatives, isomers, racemates, diastereoisomers, prodrugs, hydrates, esters, and/or analogs thereof. In certain aspects, the therapeutic can be a cytostatic agent, such as a limus drug. A limus drug may include one or more of sirolimus, biolimus (biolimus A9), everolimus, zotarolimus, and tacrolimus. The therapeutic agent may be an anti-fibrotic drug. Anti-fibrosis pharmacological mechanisms of action include reduction in local fibroblast proliferation, reduction in local inflammation, and reductions in fibrous tissue growth factors. Anti-fibrotic drugs include, for example, triamciclone, tranilast, halofuginone, montelukast, zafirlukast, pirfenidone and nintedanib. For example, therapeutic agents such as pirfenidone and nintedanib may slow the progression of scar tissue build up.

Other drugs that may be useful in the present disclosure include, without limitation, glucocorticoids (e.g., cortisol, betamethasone), hirudin, angiopeptin, aspirin, growth factors, antisense agents, anti-cancer agents, anti-proliferative agents, oligonucleotides, and, more generally, anti-platelet agents, anti-coagulant agents, anti-mitotic agents, antioxidants, anti-metabolite agents, anti-chemotactic, and anti-inflammatory agents. Also useful in aspects of the present disclosure are polynucleotides, antisense, RNAi, or siRNA, for example, that inhibit inflammation and/or smooth muscle cell or fibroblast proliferation, contractility, or mobility. Anti-platelet agents can include drugs such as aspirin and dipyridamole. Aspirin is classified as an analgesic, antipyretic, anti-inflammatory and anti-platelet drug. Dipyridamole is a drug similar to aspirin in that it has anti-platelet characteristics. Dipyridamole is also classified as a coronary vasodilator. Anti-coagulant agents for use in aspects of the present disclosure can include drugs such as heparin, protamine, hirudin and tick anticoagulant protein. Anti-oxidant agents can include probucol. Anti-proliferative agents can include drugs such as amlodipine and doxazosin. Anti-mitotic agents and anti-metabolite agents that can be used in aspects of the present disclosure include drugs such as methotrexate, azathioprine, vincristine, adriamycin, and mutamycin. Antibiotic agents for use in aspects of the present disclosure include penicillin, cefoxitin, oxacillin, tobramycin, and gentamicin. Suitable antioxidants for use in aspects of the present disclosure include probucol. Additionally, genes or nucleic acids, or portions thereof can be used as the therapeutic agent in aspects of the present disclosure. Photosensitizing agents for photodynamic or radiation therapy, including various porphyrin compounds such as porfimer, for example, are also useful as drugs in aspects of the present disclosure.

A combination of drugs can also be used in some aspects of the present disclosure. Some of the combinations have additional effects because they have a different mechanisms. In aspects, the additional effects may be advantageous for use in the drug coatings described herein. For example, in some aspects, because of the additional effects, the dose of the drug can be reduced. In aspects, combinations of therapeutic agents may reduce complications from using a high dose of the therapeutic agent.

In some aspects, the encapsulation layer may include one or more polymer additives. In some aspects, the additives provide additional properties to the polymers. In some aspects, the polymer additive(s) may include glycerol, a poly ethylene glycol, sorbitol, a polysorbate, and/or multi-functional cross-linkers. In some aspects, the polymer additives are hydrophilic in nature and contribute to the absorption and/or retention of aqueous media within the encapsulation layer. Through the polymer, optionally in conjunction with the polymer additives, the encapsulation layer swells sufficiently to fill the cavity or space within the subject. The swelling of the encapsulation layer further allows for contrast with the permanent or semi-permanent material such that when an imaging modality is applied to the subject, the occupation of the full cavity allows for the tissue marker to be easily detected.

In some aspects, the one or more components may include at least one excipient, such as a surfactant, an antioxidant, a vitamin, or combinations thereof. An excipient may be a biodurable polymer. As set forth herein, a biodurable polymer may include a polymer that is well-tolerated and/or non-reactive when contacted to a subject or immune-reactive cells thereof and is resistant to erosion and/or enzymatic degradation and/or dissolution within the subject or the circulatory system thereof. Biodurable polymers include polyethylene terephthalate (PET), nylon 6,6, polyurethane (PU), polytetrafluoroethylene (PTFE), polyethylene (PE, low density and high density and ultra-high molecular weight, UHMW), polysiloxanes (silicones) and poly(methylmethacrylate) (PMMA) and Poly(vinylidene fluoride-co-hexafluoropropylene) (PVDF-HFP). In some aspects, the excipient may be PVDF-HFP. Many other excipients may be useful for purposes of the present disclosure, such as polyglutamic acid, polyacrylic acid, hyaluronic acid, alginate, PVA, PVP, Pluronic (PEO—PPO-PEO), cellulose, CMC, HPC, starch, chitosan, human serum albumin (HSA), phospholipids, fatty acid, fatty acid esters, triglycerides, beeswax, cyclodextrin, polysorbates, polyethylene glycol, polyvinylpyrrolidone (PVP) and aliphatic polyesters. One or more excipients may be selected from amino alcohols, alcohols, amines, acids, amides and hydroxyl acids in both cyclo- and linear-aliphatic and aromatic groups. Examples include L-ascorbic acid and its salt, D-glucoascorbic acid and its salt, tromethamine, triethanolamine, diethanolamine, meglumine, glucamine, sodium docusate, urea, amine alcohols, glucoheptonic acid, glucomic acid, hydroxyl ketone, hydroxyl lactone, gluconolactone, glucoheptonolactone, glucooctanoic lactone, gluconic acid lactone, mannonic lactone, ribonic acid lactone, lactobionic acid, glucosamine, glutamic acid, benzyl alcohol, benzoic acid, hydroxybenzoic acid, propyl 4-hydroxybenzoate, lysine acetate salt, gentisic acid, lactobionic acid, lactitol, sorbitol, glucitol, sugar phosphates, glucopyranose phosphate, sugar sulphates, sugar alcohols, sinapic acid, vanillic acid, vanillin, methyl paraben, propyl paraben, xylitol, 2-ethoxyethanol, sugars, galactose, glucose, ribose, mannose, xylose, sucrose, lactose, maltose, arabinose, lyxose, fructose, cyclodextrin, (2-hydroxypropyl)-cyclodextrin, acetaminophen, ibuprofen, retinoic acid, lysine acetate, gentisic acid, catechin, catechin gallate, tiletamine, ketamine, propofol, lactic acids, acetic acid, salts of any organic acid and amine described above, polyglycidol, glycerol, multiglycerols, galactitol, di(ethylene glycol), tri(ethylene glycol), tetra(ethylene glycol), penta(ethylene glycol), di(propylene glycol), tri(propylene glycol), tetra(propylene glycol, and penta(propylene glycol), and combinations thereof. Excipients may be selected from amino acids and salts thereof. For example, the excipient may be one or more of alanine, arginine, asparagines, aspartic acid, cysteine, cystine, glutamic acid, glutamine, glycine, histidine, proline, isoleucine, leucine, lysine, methionine, phenylalanine, serine, threonine, tryptophan, tyrosine, valine, and derivatives thereof are. Certain amino acids, in their zwitterionic form and/or in a salt form with a monovalent or multivalent ion, have polar groups, relatively high octanol-water partition coefficients, and are useful in some facets of the present disclosure. The excipient may include a surfactant: a chemical compound with one or more hydroxyl, amine, carbonyl, carboxyl, amides or ester moieties; or both. Exemplary surfactants may be chosen from PEG fatty esters, PEG omega-3 fatty esters and alcohols, glycerol fatty esters, sorbitan fatty esters, PEG glyceryl fatty esters, PEG sorbitan fatty esters, sugar fatty esters, PEG sugar esters, Tween 20, Tween 40, Tween 60, p-isononylphenoxypolyglycidol, PEG laurate, PEG oleate, PEG stearate, PEG glyceryl laurate, PEG glyceryl oleate, PEG glyceryl stearate, polyglyceryl laurate, polyglyceryl oleate, polyglyceryl myristate, polyglyceryl palmitate, polyglyceryl-6 laurate, polyglyceryl-6 oleate, polyglyceryl-6 myristate, polyglyceryl-6 palmitate, polyglyceryl-10 laurate, polyglyceryl-10 oleate, polyglyceryl-10 myristate, polyglyceryl-10 palmitate, PEG sorbitan monolaurate, PEG sorbitan monolaurate, PEG sorbitan monooleate, PEG sorbitan stearate, PEG oleyl ether, PEG laurayl ether, Tween 20, Tween 40, Tween 60, Tween 80, octoxynol, monoxynol, tyloxapol, sucrose monopalmitate, sucrose monolaurate, decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonyl-β-D-glucopyranoside, octanoyl-N-methylglucamide, n-octyl-β-D-glucopyranoside, octyl-β-D-thioglucopyranoside and their derivatives. In some aspects, the excipients may include one of sodium docusate sorbitol, urea, BHT, BHA, PEG-sorbitan monolaureate, petrolatum, methyl stearate or a combination thereof. Antioxidants that may be used in the present disclosure include, without limitation, oligomeric or polymeric proanthocyanidins, polyphenols, polyphosphates, polyazomethine, high sulfate agar oligomers, chitooligosaccharides obtained by partial chitosan hydrolysis, polyfunctional oligomeric thioethers with sterically hindered phenols, hindered amines such as, without limitation, p-phenylene diamine, trimethyl dihydroquinolones, and alkylated diphenyl amines, substituted phenolic compounds with one or more bulky functional groups (hindered phenols) such as tertiary butyl, arylamines, phosphites, hydroxylamines, and benzofuranones. Also, aromatic amines such as p-phenylenediamine, diphenylamine, and N,N′ disubstituted p-phenylene diamines may be utilized as free radical scavengers. Other examples include, without limitation, butylated hydroxytoluene (“BHT”), butylated hydroxyanisole (“BHA”), L-ascorbate (Vitamin C), Vitamin E, herbal rosemary, sage extracts, glutathione, resveratrol, ethoxyquin, rosmanol, isorosmanol, rosmaridiphenol, propyl gallate, gallic acid, caffeic acid, p-coumeric acid, p-hydroxy benzoic acid, astaxanthin, ferulic acid, dehydrozingerone, chlorogenic acid, ellagic acid, propyl paraben, sinapic acid, daidzin, glycitin, genistin, daidzein, glycitein, genistein, isoflavones, and tertbutylhydroquinone. Examples of some phosphites include di(stearyl)pentacrythritol diphosphite, tris(2,4-di-tert.butyl phenyl)phosphite, dilauryl thiodipropionate and bis(2,4-di-tert.butyl phenyl)pentaerythritol diphosphite. Some examples, without limitation, of hindered phenols include octadecyl-3,5,di-tert.butyl-4-hydroxy cinnamate, tetrakis-methylene-3-(3′,5′-di-tert.butyl-4-hydroxyphenyl)propionate methane 2,5-di-tert-butylhydroquinone, ionol, pyrogallol, retinol, and octadecyl-3-(3,5-di-tert.butyl-4-hydroxyphenyl)propionate. An antioxidant may include glutathione, lipoic acid, melatonin, tocopherols, tocotrienols, thiols, Beta-carotene, retinoic acid, cryptoxanthin, 2,6-di-tert-butylphenol, propyl gallate, catechin, catechin gallate, and quercetin.

### Coating Layer

In some aspects, the tissue marker of the present disclosure includes a coating layer that at least in part covers the encapsulation layer and/or the permanent/semi-permanent material. In some aspects, the coating layer fully covers the encapsulation layer and/or permanent/semi-permanent material. In some aspects, the encapsulation layer may fully cover the permanent/semi-permanent material and the coating layer fully covers the encapsulation layer, thereby provide at least three layers within the tissue marker. The coating layer is designed to be of a material that dissolves and/or erodes following placement of the tissue marker in situ within a subject. In some aspects, the coating layer is of two or more materials that dissolve or naturally erode within a subject. It is an object of the coating layer to protect the encapsulation layer from immediate swelling to allow for the cavity being filled to be sealed. It will be appreciated that premature swelling can frustrate procedures such as suturing the subject.

In some aspects, the coating layer may include a hydrophobic polymer, a protein, a peptide, a hydrophobic small molecule, or a combination thereof. The presence of polymer(s) and/or protein(s) in the coating layer further provide for protection against the tissue marker migrating from the position of placement within the subject and/or the extrusion of the tissue marker in the subject.

In some aspects, the coating layer includes a base material that provides resistance from water or water-based solutions reaching the encapsulation layer. Such may include one or more of a purified starch, such as a plant starch (e.g. Arista™), chitosan, hyaluronic acid (HA), carboxymethyl cellulose (CMC), an HA-CMC co-polymer, beeswax, paraffin wax, glycerol, polyethylene glycol (PEG), sorbitol, polysorbates (such as TWEEN), dextran sulfate, pectin, pullulan, hydroxypropyl methyl cellulose (HPMC), sodium alginate, and polyvinyl alcohol (PVA). In some aspects, the coating layer is hydrophobic or predominantly hydrophobic to protect the encapsulation layer from premature swelling. Additional materials that may be included in the coating layer include, but are not limited to, hydrophobic polymers and/or hydrophobic small molecules and/or mixtures of two or more hydrophobic materials. By way of example and not limitation, examples of suitable materials include semi-synthetic glycerides (e.g. Suppocire AIML, AML, BML, BS2, BS2X, NBL, NAIS 10, CS2X), lecithin, hydrogenated coconut oil, coconut oil, cocoa butter, glycerinated gelatin, hydrogenated vegetable oils, hard fats, mineral oil, cetyl alcohol, petrolatum, petroleum jelly, decanol, soft paraffin, tridecanol, dodecanol, long chain saturated fatty acids, long chain unsaturated fatty acids, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, fatty acid triglycerides, fatty acid alcohols, PEG-fatty acid esters (with hydrophilic-lipophilic balance (HLB) below 13), PEG-surfactants with an HLB below 13, or combinations thereof.

In some aspects, the coating layer may include one or more agents to assist in healing the site of insertion of the tissue marker. Such agents may include tissue sealants, coagulation stimulants, hemostatic agents and combinations thereof. By way of example, such may include a fibrin glue, gelatin-based sealants, oxidized methylcellulose, glutaraldehyde-based adhesives, fibrinogen, thrombin, a fibrinogen-thrombin fleece, recombinant factor VII, secirin, dried plasma, tranexamic acid, dried platelets, platelet substitutes, aprotinin, nafamostat mesilate, epsilon-aminocaproic acid, vasopressin or vasopressin analogues, estrogen, ethamsylate, aluminum sulfate, iron (III) sulfate, aluminum chloride, and the like. The choice of agent(s) to include in the coating layer may be dependent on the tissue(s) or region where the tissue marker is to be deployed.

In some aspects, the coating layer includes a hemostatic polymer. In some aspects, the coating layer is of a plant starch, hyaluronic acid (HA), carboxymethyl cellulose (CMC), an HA-CMC co-polymer, or combinations thereof. A plant starch may include a purified plant starch or a polymer of plant carbohydrates. A plant starch may include a combination of branched amylopectins and helical amylose. In some aspects, the plant starch may include the product Arista™. In some aspects, the hemostatic polymer may include microporous polysaccharide hemispheres derived from purified plant starch.

In some aspects, the coating layer includes a mixture of a plant starch and an HA-CMC copolymer. In some aspects, the coating layer includes a first sub-part that includes a plant starch and an HA-CMC copolymer, such as microporous polysaccharide hemispheres and an HA-CMC copolymer. In some aspects, the first sub-part includes about 1 to 10 wt % microporous polysaccharide hemispheres, including about 2, 3, 4, 5, 6, 7, 8, and 9 wt % microporous polysaccharide hemospheres. In some aspects, the first sub-part includes about 0.5 to about 3 wt % HA-CMC copolymer including about 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, and 2.9 wt % HA-CMC copolymer. In some aspects, the coating layer includes a second sub-part of chitosan, such as a high molecular weight (HMW) chitosan, of about 0.5 to about 3 wt % chitosan including about 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, and 2.9 wt % chitosan. In some aspects, the first sub part is of about 10 to about 75 wt % of the coating layer, including about 15, 20, 25, 30, 35, 40, 45, 50, 55, 60, 65, and 70 wt %. In some aspects, the second sub part is of about 10 to about 75 wt % of the coating layer, including about 15, 20, 25, 30, 35, 40, 45, 50, 55, 60, 65, and 70 wt %.

It will be appreciated that the coating layer may also include additional components such as those described with the encapsulation layer, including excipients, therapeutic agents, therapeutic agents within micelles, and/or therapeutics within microparticles.

Various techniques may be used for applying the coating layer, as either a coating solution or a coating mixture, to the encapsulation layer, such as metering, casting, spinning, spraying, dipping (immersing), rolling, ink jet printing, 3D printing, electrostatic techniques, plasma etching, vapor deposition, and combinations of these processes. Choosing an application technique principally depends on the viscosity and surface tension of the coating solution or coating mixture. In some aspects of the present disclosure, metering, dipping and spraying may be preferred because it makes it easier to control the uniformity of the thickness of the coating layer as well as the concentration of any therapeutic agent present in the coating layer that is applied to the encapsulation layer. Regardless of whether the coating layer is applied by spraying or by dipping or by another method or combination of methods, additional coating layers may be applied to the encapsulation layer in multiple application steps in order to control the uniformity and/or the amount of therapeutic substance and additive applied to the tissue marker.

In some aspects, the coating layer may provide a low friction surface to function to provide smooth deployment of the tissue marker during placement in situ.

In some aspects, the coating layer and the encapsulation layer are both biodegradable. In some aspects, the coating layer degrades at a different rate than the encapsulation layer. In some aspects, the coating layer may need to at least partially degrade to expose the encapsulation layer to allow the encapsulation layer to degrade. In some aspects, the coating layer may degrade and/or dissolve when placed in situ within a subject. It will be appreciated that the permanent or semi-permanent detectable marker resides within the subject in situ for a prolonged period of time after the coating layer and/or encapsulation layer have completely disintegrated and/or been absorbed by the subject. The permanent marker includes a material detectable by an imaging modality thereby allowing for identification of the site where the tissue marker was inserted within the subject long after healing. The combination of the material of the encapsulation layer and the slowed expansion the cross-linked polymers therein may result in the coating layer promoting cell adhesion and subsequently allowing the encapsulation layer to function as a scaffold for cell growth to prevent migration from the implantation location. The coating layer may also serve to prevent the tissue marker from extruding prematurely from a means of implantation such as a syringe. In some aspects, the coating layer may break or rupture as the tissue marker is maneuvered into the cavity within the subject. Once the coating layer breaks or starts to dissolve, the encapsulation layer will begin to expand as a result of uptake of the fluid inherently present at the target site. The coating layer will continue to break or crack as the encapsulation layer increases in size due to swelling.

### Assembled Tissue Markers

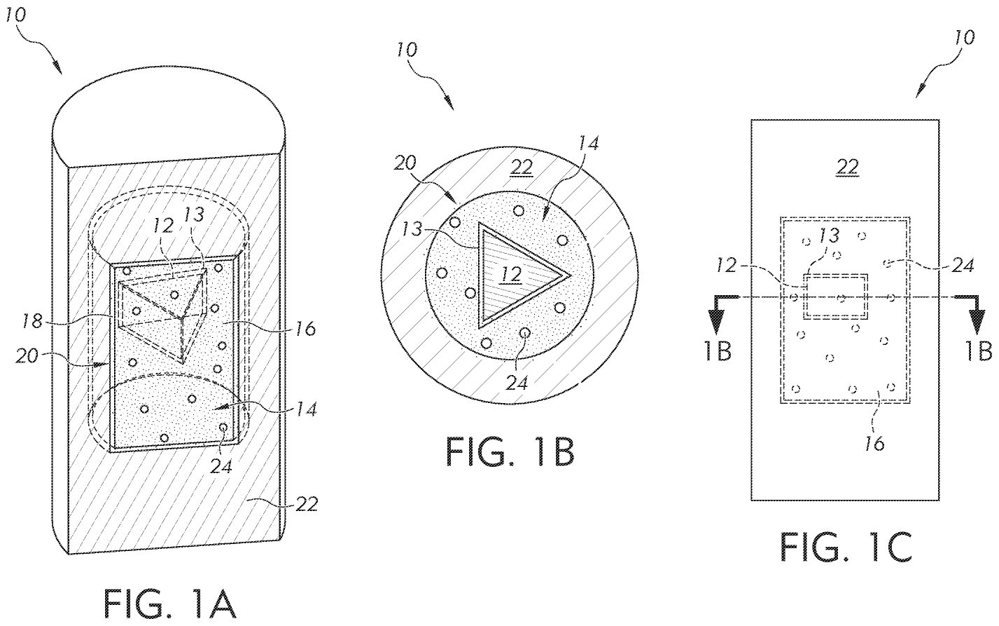

Referring now to the drawings, and more particularly to FIGS. 1A-1C, there is shown a tissue marker 10 that generally includes a permanent or semi-permanent material 12 and an encapsulation layer 14. The permanent or semi-permanent material 12 is resistant to degradation over time and may include a material detectable by an imaging modality, such as a metallic material, compound, amalgam, alloy, or the like, such as, for example, titanium, nitinol, or other biocompatible metals or metal alloys that are detectable by an imaging modality, such as, but not limited to, ultrasound imaging, fluoroscopy/X-ray, magnetic resonance imaging (MRI), or the like. The permanent or semi-permanent material 12 may have modified surface properties, such as, a non-smooth or bumpy surface. For example, the permanent or semi-permanent material 12 may include one or more metallic materials (metallic forms or metallic shapes) that are made from multiple strands of steel that are layered or coiled together to create a textured surface. Textured, non-smooth, or bumpy surface properties may allow for enhanced integration to the host and aid against migration from the cavity once the encapsulation layer 14 has degraded. The permanent or semi-permanent material 12 may include one or more metallic objects, each made of one or more metallic materials, and these metallic objects may come in various shapes or forms.

Furthermore, in some aspects, the permanent or semi-permanent material 12 is covered with a polymer protective material 13. In such aspects, the polymer protective material 13 may be a composition selected from fibroin, chitosan, carboxylmethyl cellulose (i.e. CMC), polyglycerol sebacate, or hyaluronic acid. Various techniques may be used for applying the polymer protective material 13, which may be a coating solution or a coating mixture, to the permanent or semi-permanent material 12 such as metering, casting, spinning, spraying, dipping (immersing), rolling, ink jet printing, 3D printing, electrostatic techniques, plasma etching, vapor deposition, and combinations of these processes. Choosing an application technique principally depends on the viscosity and surface tension of the polymer protective material 13. Additional coating layers may be applied to the permanent or semi-permanent material 12 in multiple application steps in order to control the uniformity of the polymer protective material 13, to control the amount of therapeutic substance and additive included in the polymer protective material 13, or both as the polymer protective material 13 is applied to the permanent or semi-permanent material 12.

The encapsulation layer 14 generally surrounds or encapsulates the permanent or semi-permanent material 12. Stated differently, the permanent or semi-permanent material 12 is generally entirely contained within the encapsulation layer 14. The permanent or semi-permanent material 12 is suspended within the encapsulation layer 14. In embodiments, the tissue marker 10 is delivered to a tissue in a patient through ejection from an introducer, such as, a biopsy cannula or a delivery tube. In the ensuing discussion, the biopsy and treatment site described will generally be the human breast, although the present disclosure is suitable for marking biopsy sites in other parts of the human and other mammalian body as well.

The encapsulation layer 14 may generally include a cross-linked biodegradable polymer 16 having a surface interface 18 with a coating layer 22. The coating layer 22 may cover the outer surface of the encapsulation layer 14. Alternatively, the coating layer 22 may be cross-linked with the cross-linked biodegradable polymer 16 of the encapsulation layer 14. Each of the coating layer 22 and the encapsulation layer 14 may include additives such as excipients, therapeutic agents, surfactants, and hemostatic agents as described herein.

The coating layer 22, covering the encapsulation layer 14, may be made of, but is not limited to, polymethyl methacrylate (PMMA), polyimide, beeswax, paraffin wax, glycerol, polyethylene glycol (PEG), sorbitol, polysorbate, dextran sulfate, pectin, pullulan, hydroxypropyl methyl cellulose (HPMC), sodium alginate, polyvinyl alcohol (PVA), or a combination of thereof. Hemostatic agents which may be included in the coating to promote blood coagulation to seal the cavity may include, but are not limited to, fibrinogen, sericin, or the like. The coating layer 22 may provide the tissue marker 10 with the following advantages: (1) smooth deployment from an introducer cannula via the coating layer 22 providing a low friction external surface; (2) reduction or elimination of instances of partial deployment, i.e., early or immediate expansion of the encapsulation layer 14 as soon as the tissue marker 10 is first introduced to the target site in the tissue of the patient, by slowing the expansion/hydration of the encapsulation layer 14 by the coating layer 22 functioning as a barrier between the fluids inherently present at the target site and the encapsulation layer 14, which may have hemostatic properties; (3) prevention of the tissue marker 10 from temporarily sticking to its introducer cannula (not shown) during delivery by preventing the early expansion/hydration of the encapsulation layer 14 by the coating layer 22 functioning as a barrier between any fluid in the introducer and the encapsulation layer 14; and (4) promotion of a coagulation effect by including a hemostatic agent in the composition of the coating layer 22.

In aspects, the encapsulation layer 14 may include a plurality of microparticles, therapeutics and/or excipients 24 dispersed in the cross-linked biodegradable polymer 16, which will be described in greater detail below.

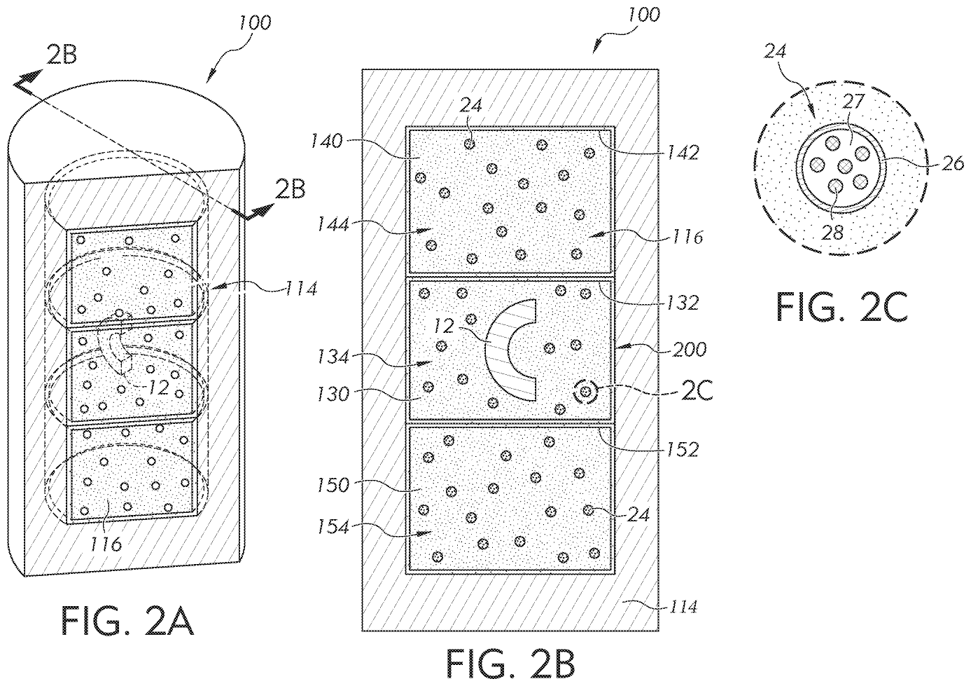

Referring now to FIGS. 2A-2B, the embodiment of housing the permanent/semi-permanent material 12 within the tissue marker 100 is depicted The tissue marker 100 includes an encapsulation layer 114 surrounding a housing compartment 116, with a permanent or semi-permanent material 12 enclosed therein (coating layer not depicted here). Accordingly, the description above with respect to such features are applicable unless otherwise noted and will not be repeated.

The encapsulation layer 114 surrounds an outer surface 200 of the housing compartment 116. In the depicted embodiment, the housing compartment 116 is formed into two or more compartments. One or more walls partition the housing compartment 116 into the two or more compartments. The one or more walls that partition the housing compartment 116 are made of the same material as the housing compartment 116, but the one or more walls have a different molecular density than the housing compartment 116. In the embodiment of FIGS. 2A-2C, the tissue marker 100 is shown having an encapsulation layer 114 surrounding a first compartment 130, a second compartment 140, and a third compartment 150. However, the housing compartment 116 may have any number of compartments.

The first compartment 130 includes a first protective polymer wall 132 that entirely surrounds a first interior 134 of the first compartment 130. The second compartment 140 includes a second protective polymer wall 142 that entirely surrounds a second interior 144 of the second compartment 140. The third compartment 150 includes a third protective polymer wall 152 that entirely surrounds a third interior 154 of the third compartment 150. In embodiments, the first compartment 130 encapsulates the permanent or semi-permanent material 12 of the tissue marker 100, though it is contemplated that any or all of the first compartment 130, the second compartment 140, or the third compartment 150 could encapsulate a permanent/semi-permanent material 12.

Each of the first compartment 134, the second compartment 140, and the third compartment 150 is configured with a hydrogel 115 to receive a therapeutic agent or two or more thereof. In embodiments, the therapeutic agent is delivered to the tissue marker 100 inside a micelle 24 or as part of a microparticle (not depicted).

Referring to FIG. 2C, which is an enlargement from FIG. 2B, each of the micelle 24, which may also be present in the embodiment of FIGS. 1A-1C, includes at least one outer lipid membrane 26, a core 27, and the therapeutic agent 28 within the lipid membrane 26. In embodiments, the size of each of the micelles 24 is in the range of 10 micrometers up to and including 200 micrometers. In some embodiments, the average size of each of the distinct structures 24 is 1.1 micrometers plus or minus 0.39 micrometers. In some embodiments, the average diameter of the plurality of distinct structures 24 is from 60 nanometers to 70 nanometers.

The degradation of the lipid membrane 26 is triggered by an external energy source, such as, focused ultrasound from the application of non-invasive focused ultrasound. Thus, the lipid membrane 26 is configured to open upon the application of non-invasive focused ultrasound to release the therapeutic agent 28 from within the micelle 24 and into the hydrogel 115. Ultimately, after non-invasive focused ultrasound has been applied to the lipid membrane 26, the therapeutic agent 28 diffuses from the interior of the distinct structure 24 to the hydrogel 116 and then diffuses from the housing compartment 116 into the encapsulation layer 114 and ultimately to a tissue of a patient.

According to some embodiments, the therapeutic agent is not encased in a micelle or a microparticle, but instead is dispersed directly into the encapsulation layer 114, the hydrogel 115 or an interior of a compartment, such as the second interior 144 of second compartment 140, as shown in FIG. 2B. In such embodiments, the therapeutic agent is released to the tissue via linear diffusion.

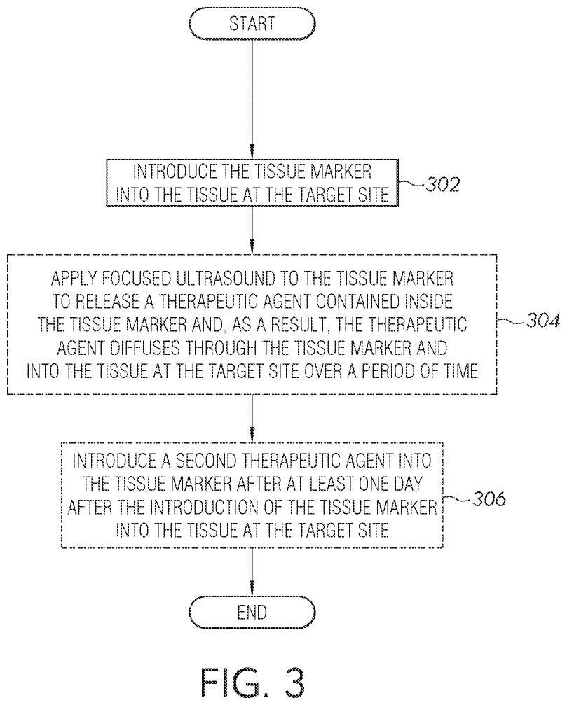

FIG. 3 is a flowchart depicting a specific method of introducing the therapeutic agent into a tissue at a target site. The therapeutic agent includes, but is not limited to, drugs, antibiotics, cancer chemotherapy agents, hormones, anesthetic agents, and other medicinal compounds. The method may include a greater or fewer number of steps in any order without departing from the scope of the present disclosure. For example, in embodiments, the method may include providing a tissue marker to a target site (e.g., a biopsy site).

The method further includes at step 302 introducing the tissue marker into the tissue at the target site. The method of introducing as shown in FIG. 3 may be conducted by syringe or a similar introducer.

Since the lipid membrane prevents the therapeutic agent from being released, the method of FIG. 3 may include step 304, applying non-invasive, focused ultrasound from a probe held external to the patient's body. Stated differently, the lipid membrane is burst by non-invasive, focused ultrasound applied non-invasively by a probe that remains external to the patient. Step 304 of applying focused ultrasound releases the therapeutic agent and, as a result, the therapeutic agent then diffuses into the tissue at the target site over a period of time. Furthermore, the diffusion may take place over a period of time as the encapsulation layer may be degraded/resorbed by the biological environment of the target site.

In addition or in alternative to step 304 described above, the step of applying non-invasive, focused ultrasound may be directed to at least one wall to burst the wall of the housing compartment. For instance, after at least third wall has been burst via application of non-invasive, focused ultrasound, the therapeutic agent within the third compartment will diffuse into the tissue in which the tissue marker has been introduced, as a result of step 302.

In addition, the method of FIG. 3 may include step 306, introducing a second therapeutic agent into the housing compartment and/or encapsulation layer. For example, the second therapeutic agent may be introduced into the hydrogel and/or encapsulation layer at a point in time (e.g., at least one day) after implantation of the marker into the patient. For example, the second therapeutic agent may be introduced into the hydrogel and/or encapsulation layer via syringe by introducing micelles loaded with a therapeutic, microparticles containing a therapeutic, amorphous therapeutic suspended or dissolved in solution and/or crystalline therapeutic suspended in solution. The second therapeutic agent may have the same composition or a different composition as the first therapeutic agent that was included when the marker was first introduced to the target site. For example, in some embodiments, the first therapeutic agent is an antibiotic, and the second therapeutic agent delivered days or weeks later is a chemotherapy agent. In another example, the first therapeutic agent includes an antibiotic and a first chemotherapy agent, such as, doxorubicin, and after observing the progress of the patient over time, such as two weeks, the physician determines that the introduction of the second therapeutic agent, such as, a second chemotherapy agent, such as, paclitaxel, which is, of course, different compositionally from the first therapeutic agent, is to be delivered into one or more of the plurality of compartments of the tissue marker that was previously placed in vivo into the tissue at the target site.

The method of FIG. 3 may be performed as part of a biopsy procedure, for example, after a tissue sample has been taken, the tissue marker may be introduced into the subject tissue at the target site. Alternatively, the method of FIG. 3 may be performed on its own or in conjunction with other invasive procedures.

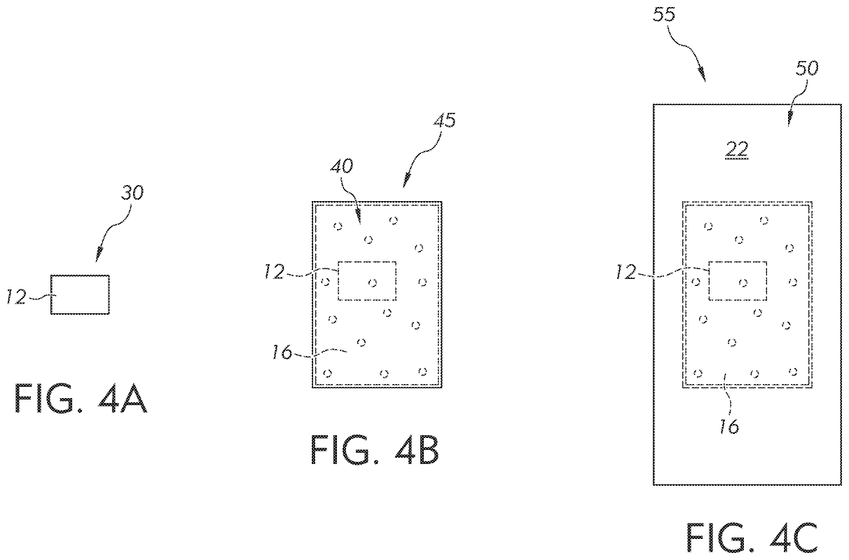

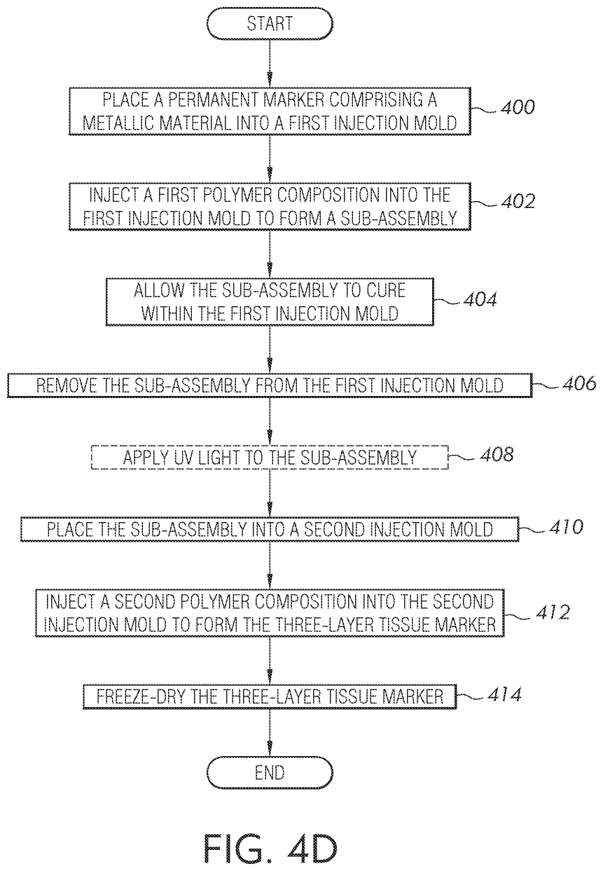

The tissue marker 10 of FIGS. 1A-1C is a three-layer tissue marker 55 that has a first layer 30, a second layer 40, and third layer 50, which are depicted in FIGS. 4A-4C, respectively. At the core of the tissue marker 10 is the first layer 30, which is the permanent or semi-permanent material 12 described above. Covering over the first layer 30 is the second layer 40, which is the encapsulation layer 16 described above. Covering over the second layer 40 is the third layer 50, which is the coating layer 22 described above.

A method of fabricating the three-layer tissue marker, such as the tissue marker, is generally illustrated by the flowchart of FIG. 4D. It is noted that the method may be used in manufacturing of any of the markers as described herein. The method may include a greater or fewer number of steps in various orders without departing from the scope of the present disclosure.

With reference also to FIGS. 4A-4C, at step 400, the method of fabricating the three-layer tissue marker 55 may include placing the permanent or semi-permanent material 12, as described above, into a first injection mold. The permanent or semi-permanent material 12 is the first layer 30 of the three-layer tissue marker 55.

The method includes step 402 injecting a first polymer composition into the first injection mold to form the second layer 40, such as illustrated in FIG. 4B, around the first layer 30. Typically, the second layer 40 entirely encapsulates the first layer 30. The first polymer composition may be any of the compositions that may be used to form the polymer body 16 as described above with respect to FIGS. 1A-1C. Together the first layer 30 and the second layer 40 form a sub-assembly 45, as shown in FIG. 4B.

Still referring to FIGS. 4A-4D, at step 404, the second layer is allowed to cure within the first injection mold. Curing may occur by allowing time to pass. At step 406, the sub-assembly 45 of the first layer 30 and second layer 40 is removed from the first injection mold. In some embodiments, as shown at step 408, ultraviolet, i.e., UV, light may be applied to the sub-assembly 45 before placing the sub-assembly 45 into the second injection mold.

At step 410, the method may include placing the sub-assembly 45 of the first layer 30 and second layer 40 into the second injection mold.

At step 412, the method includes injecting a second polymer composition into the second injection mold to form the three-layer tissue marker 55. Step 412 of injecting the second polymer composition forms a third layer 50, as shown in FIG. 4C, around the second layer 40. The three-layer tissue marker 55 may then be allowed to cure in the second injection mold. The second polymer composition may be any of the compositions that may be used to form the coating layer. Typically, the third layer 50 entirely encapsulates the second layer 40.

In some embodiments, at step 414, the method may include freeze-drying the three-layer tissue marker 55.

### EXAMPLES

The following examples are offered by way of illustration only. In view of the foregoing description, a person having ordinary skill in the art will recognize that the following examples are not intended to limit the scope of this disclosure or its many embodiments.

### Example 1

A lyophilized foam was prepared according to the composition provided in Table 1, herein “Composition 1”.

TABLE 1
Lyophilized Foam Composition 1

| Percentage of | | | |
|---|---|---|---|
| Composition | Ingredient | W/V % | Function |
| 50% | absorbable | 5% | hemostatic and expansion |
| | microporous | | properties |
| | polysaccharide | | |
| | hemospheres | | |
| | hyaluronic acid and | 1.2% | |
| | carboxylmethyl | | |
| | cellulose | | |
| 50% | HMW Chitosan | 1% | hemostatic and expansion |
| | | | properties |

### Example 2

A lyophilized foam was prepared according to the composition provided in Table 2, herein “Composition 2”.

TABLE 2
Lyophilized Foam Composition 2

| Percentage of | | | |
|---|---|---|---|
| Composition | Ingredient | W/V % | Function |
| 40% | absorbable | 5% | hemostatic and expansion |
| | microporous | | properties |
| | polysaccharide | | |
| | hemospheres | | |
| | hyaluronic acid and | 1.2% | |
| | carboxylmethyl | | |
| | cellulose | | |
| 60% | HMW Chitosan | 1% | hemostatic and expansion |
| | | | properties |

### Example 3

A lyophilized foam was prepared according to the composition provided in Table 3, herein “Composition 3”.

TABLE 3
Lyophilized Foam Composition 3

| Percentage of | | | |
|---|---|---|---|
| Composition | Ingredient | W/V % | Function |
| 60% | absorbable | 5% | hemostatic and expansion |
| | microporous | | properties |
| | polysaccharide | | |
| | hemospheres | | |
| | hyaluronic acid and | 1.2% | |
| | carboxylmethyl | | |
| | cellulose | | |
| 40% | HMW Chitosan | 1% | hemostatic and expansion |
| | | | properties |

### Example 4

A lyophilized foam was prepared according to the composition provided in Table 4, herein “Composition 4”.

TABLE 4
Lyophilized Foam Composition 4

| Percentage of | | | |
|---|---|---|---|
| Composition | Ingredient | W/V % | Function |
| 30% | absorbable | 5% | hemostatic and expansion |
| | microporous | | properties |
| | polysaccharide | | |
| | hemospheres | | |
| | hyaluronic acid and | 1.2% | |
| | carboxylmethyl | | |
| | cellulose | | |
| 70% | HMW Chitosan | 1% | hemostatic and expansion |
| | | | properties |

### Experimental Procedures

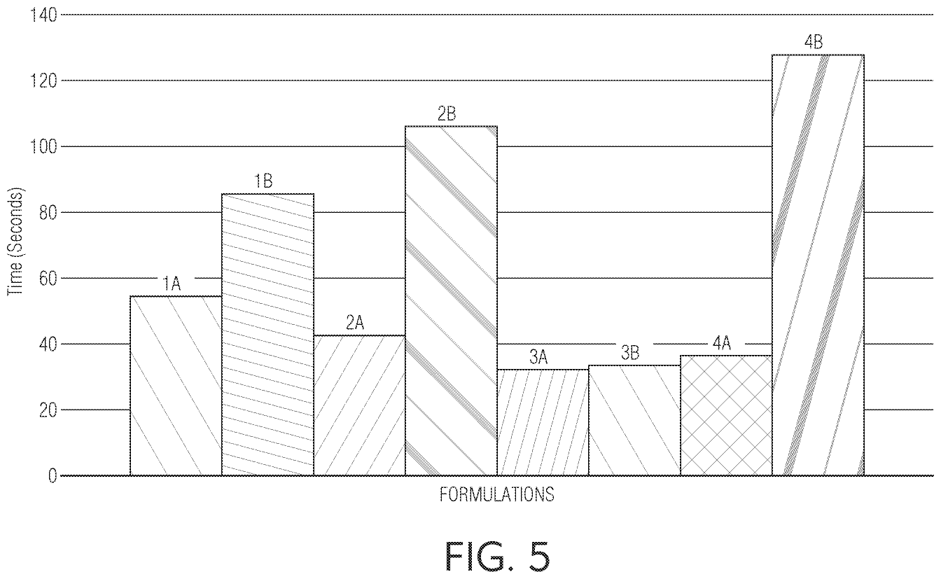

Referring now to FIG. 5, an experiment was conducted to determine certain materials and chemical compositions and certain molecular weights would have improved hemostatic properties for the coating 22 of tissue marker 10 or the tissue marker 100. Bovine blood was purchased from Animal Technologies, Inc. (Tyler TX) with sodium citrate added as an anticoagulant agent.

Before experimentation, the blood was neutralized. The process of neutralizing the blood included: bringing the blood to room temperature, such as, by allowing the blood to rest for three hours at room temperature. Next, 60 mL of blood was poured into a 150 ml beaker. Then, 0.1476 g CaCl₂) was added to the blood and stirred for 30 minutes at room temperature.

Next, the blood clotting time was measured for each formulation reported in FIG. 5 and Table 5. A first formulation “A” of each exemplary composition of lyophilized foam of absorbable microporous polysaccharide hemospheres, high molecular weight (HMW) chitosan, hyaluronic acid and carboxylmethyl cellulose was created by: placing one (1) mL of blood in a small glass vial containing 100 mg of lyophilized foam, such as, for example, Composition 1, described above. The high molecular weight chitosan was in the molecular weight range of 310,000-375,000 Daltons (Da). This step was repeated two additional times for a total of three vials for each formulation reported in FIG. 5 and Table 5. In addition, a second formulation “B” of each exemplary composition of lyophilized foam was created by mixing 1 mL of blood with 75 mg of lyophilized foam, such as, for example, Composition 1, described above. This step was repeated two additional times for a total of three vials for each formulation reported in FIG. 5 and Table 5.

The vials were placed in a 37° C. water bath. Each vial was tilted every 15 seconds until the blood clotted. The elapsed time was recorded from the time in the water bath until clotted. Each elapse time reported in the graph of FIG. 5 and Table 5 below is an average of each elapsed time of each of the three vials.

### Results

Table 5, depicted below, shows the blood clotting time of different amounts of the exemplary compositions of lyophilized foam used. FIG. 5 is a graphical representation of the data reported in Table 5. The results show blood clotting time depends on the composition of lyophilized foam and the amount of each composition.

Unexpectedly, Composition 3 provided in Example 3 demonstrated the fastest clotting time regardless of the amount of lyophilized foam added to the blood.

TABLE 5
Results

| | Lyophilized | Sample | Average | Average |
|---|---|---|---|---|
| | Foam | Weight | Clot Time | Clot Time |
| Formulations | Composition | (milligrams) | (minutes) | (seconds) |
| 1A | 1C | 100 | 0.91 | 54.4 |
| 1B | 1Composition 2 | 75 | 1.43 | 85.8 |
| 2A | 2Composition | 100 | 0.7 | 42 |
| 2B | 2 | 75 | 1.77 | 106.2 |
| 3A | 3 | 100 | 0.54 | 32.1 |
| 3B | 3 | 75 | 0.55 | 33 |
| 4A | 4 | 100 | 0.6 | 36 |
| 4B | 4 | 75 | 2.13 | 127.8 |

Thus, the experimental procedure shows that in certain embodiments, it would be advantageous for the coating 22 of tissue marker 10 or tissue marker 100, to include a 60:40 composition, in which 60% of the composition is made up of 5% absorbable microporous polysaccharide hemospheres and 1.2% hyaluronic acid and carboxylmethyl cellulose, while 40% of the composition includes high molecular weight (HMW) (1%) chitosan, for improved hemostatic properties.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

1. A tissue marker, comprising:
a permanent or semi-permanent material detectable by an imaging modality;
an encapsulation layer at least partially covering the permanent or semi-permanent material comprising a cross-linked biodegradable polymer; and
a coating layer at least partially covering the encapsulation layer comprising a hydrophobic polymer, a hydrophobic small molecule, a protein, a peptide, or a combination thereof.

2. The tissue marker of claim 1, wherein the permanent or semi-permanent material comprises a metal or metallic compound.

3. The tissue marker of claim 1, wherein the permanent or semi-permanent material is selected from titanium, nitinol, stainless steel, a cobalt-chromium alloy, a titanium alloy, a magnesium alloy, or a combination thereof.

4. The tissue marker of claim 1, wherein the cross-linked biodegradable polymer is a swellable hydrogel or swellable non-hydrogel.

5. The tissue marker of claim 1, wherein the cross-linked biodegradable polymer is selected from silk fibrin, chitosan, carboxymethyl cellulose (CMC), polyglycerol subacetate, and/or hyaluronic acid (HA).

6. The tissue marker of claim 1, wherein the coating layer is selected from a plant starch, chitosan, hyaluronic acid (HA), carboxymethyl cellulose (CMC), an HA-CMC co-polymer, beeswax, paraffin wax, glycerol, polyethylene glycol (PEG), sorbitol, a polysorbate, dextran sulfate, pectin, pullulan, hydroxypropyl methyl cellulose (HPMC), sodium alginate, polyvinyl alcohol (PVA), a glyceride, lecithin, hydrogenated coconut oil, coconut oil, cocoa butter, glycerinated gelatin, hydrogenated vegetable oils, hard fats, mineral oil, cetyl alcohol, petrolatum, petroleum jelly, decanol, soft paraffin, tridecanol, dodecanol, long chain saturated fatty acids, long chain unsaturated fatty acids, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, carnauba wax, fatty acid triglycerides, fatty acid alcohols, polyethylene glycol (PEG)-fatty acid esters, PEG-surfactants with an HLB below 13, or combinations thereof.

7. The tissue marker of claim 5, wherein the coating layer comprises at least one of as a plant starch, chitosan, hyaluronic acid (HA), carboxymethyl cellulose (CMC), and an HA-CMC co-polymer or is a combination thereof.

8. The tissue marker of claim 1, wherein the permanent or semi-permanent material further comprises a protective polymer coating.

9. The tissue marker of claim 8, wherein the protective polymer coating comprises fibroin, chitosan, carboxylmethyl cellulose (CMC), polyglycerol sebacate, hyaluronic acid (HA), or a combination thereof.

10. The tissue marker of claim 1, wherein the encapsulation layer at least partially covers a housing compartment and wherein the permanent or semi-permanent material resides within a compartment of the housing compartment.

11. The tissue marker of claim 10, wherein the housing compartment is comprised of fibroin, chitosan, carboxylmethyl cellulose (CMC), polyglycerol sebacate, and/or hyaluronic acid (HA).

12. The tissue marker of claim 11, wherein the housing compartment comprises two or more compartments.

13. The tissue marker of claim 12, wherein at least one compartment of the housing compartment comprises an interior space filled with a hydrogel.

14. The tissue marker of claim 13, wherein the hydrogel has a therapeutic agent dispersed therein.

15. The tissue marker of claim 12, wherein the hydrogel has an excipient dispersed therein.

16. The tissue marker of claim 1, wherein the coating layer comprises a hemostatic polymer.

17. The tissue marker of claim 16, wherein the hemostatic polymer comprises microporous polysaccharide hemospheres.

18. The tissue marker of claim 1, wherein the encapsulation layer further comprises a therapeutic agent.

19. The tissue marker of claim 18, wherein the therapeutic agent is present within microparticles dispersed within the encapsulation layer.

20. The tissue marker of claim 19, wherein the microspheres are comprised of poly-glycolic acid (PGA) poly-L-lactic acid (PLLA), polycaprolactone (PCL), poly-DL-lactic acid (PDLLA), poly(trimethylene carbonate) (PTMC), poly (ester amine)s (PEA), poly(para-dioxanone) (PPDO), poly-2-hydroxy butyrate (PHB), a polymer combination of lactic acid and glycolic acid, poly-lactic-co-glycolic acid (PLGA), and co-polymers thereof.

21. The tissue marker of claim 20, wherein the microparticles further comprise an antioxidant.

22. The tissue marker of claim 18, wherein the therapeutic agent is present in micelles dispersed throughout the encapsulation layer.

23. The tissue marker of claim 1, wherein the encapsulation layer further comprises an excipient.

24. The tissue marker of claim 23, wherein the excipient is an antioxidant, a surfactant, a vitamin, or a combination thereof.

25. The tissue marker of claim 23, wherein the excipient comprises a surfactant and an antioxidant.

26. The tissue marker of claim 1, wherein the coating layer comprises a first sub-part comprised of a microporous polysaccharide and an HA-CMC copolymer mixture and a second sub-part comprised of chitosan.

27. The tissue marker of claim 26, wherein the chitosan is a high molecular weight chitosan.

28. The tissue marker of claim 26, wherein the first sub-part comprises between about 10 to about 70 wt % of the coating layer.

29. A method of preparing the tissue marker of claim 1, comprising:
surrounding the permanent or semi-permanent material with the encapsulating layer;
cross-linking the encapsulating layer;
applying the coating layer to the cross-linked encapsulating layer; and
cross-linking the coating layer to form a three layer tissue marker.

30. The method of claim 29, further comprising freeze drying the three layer tissue marker.

31. The method of claim 29, comprising providing a protective polymer to the permanent or semi-permanent material prior to surrounding in the encapsulation layer.

32. The method of claim 31, wherein the protective polymer surrounds the permanent or semi-permanent material in a first compartment of a housing compartment.

33. The method of claim 32, wherein the housing compartment includes a second compartment filled with a hydrogel.

34. The method of claim 33, wherein the hydrogel comprises a therapeutic agent.

35. The method of claim 29, wherein the encapsulation layer includes a therapeutic agent.

36. A method to provide support to a recess or void in a subject, comprising providing an access point to a desired tissue space in a subject, placing the tissue marker of claim 1 in the desired tissue space, and closing the access point.

37. The method of claim 36, further comprising injecting a solution comprising a therapeutic agent into the tissue marker.
